(19) United States
(12) Patent Application Publication
(10) Pub. No.: US 2008/0074614 A1
(43) Pub. Date: Mar. 27, 2008

(54) Method and system for pupil acquisition

(76) Inventors: Richard Alan Leblanc, Clermont, FL (US); Thomas L. McGilvary, Oviedo, FL (US)

Correspondence Address: ALCON, IP LEGAL, TB4-8, 6201 SOUTH FREEWAY, FORT WORTH, TX, 76134, US

(21) Appl. No.: 11/526,547

(22) Filed: Sep. 25, 2006

(51) Int. Cl. A61B 18/18; A61B 3/10; G06G 7/48

(52) U.S. Cl. 351/205; 606/13; 703/5

(57) ABSTRACT

Embodiments of the present invention provide a method and system for automatically locating a pupil. Broadly speaking, an optical beam is scanned across the eye. In any given cycle, the reflected light from the eye is detected by a light sensor and data from the sensor processed to locate some geometric feature of the reflected light, such as the centroid. If the distance between the position of the optical beam and the geometric feature is greater than a threshold value, the system determines that the pupil has been located.

### TECHNICAL FIELD OF THE INVENTION

The present invention relates to systems and methods for eye tracking. More particularly, embodiments of the present invention relate to methods and systems for identifying and acquiring a pupil for tracking of eye movements.

### BACKGROUND OF THE INVENTION

The human eye can suffer a number of maladies causing mild deterioration to complete loss of vision. Eye glasses and contact lenses are the traditional solutions to near and far sightedness. More recently, however, photorefractive keratectomy (“PRK”), Laser-Assisted Sub-Epithelial Keratectomy (“LASEK”) and Laser-Assisted In-Situ Keratomileusis (“LASIK”) have become popular procedures to correct vision problems and reduce dependence on eyewear and contact lenses. Additional procedures have also been developed including all-femtosecond correction (“FLIVC”), Epi-LASIK, and wavefront guided PRK. In many of these procedures, a finely controlled excimer laser ablates small areas of tissue to reshape the cornea, thereby changing the characteristics of the eye to enhance vision.

During laser eye surgery, the laser must be precisely placed to achieve the desired results despite the fact that the patient's eye may be moving during the procedure. Therefore, laser surgical systems include a variety of eye tracking systems to help ensure that the laser is accurately aimed. Eye tracking systems often rely on locating and tracking a particular feature or set of features of the eye. Based on the movement of the feature or features, the eye tracking system determines the movement of the eye as a whole and adjusts the laser position accordingly. Tracking and fine adjustment of the laser can be done thousands of times a second.

Some eye tracking systems use the pupil as the tracked feature. Before tracking can begin, however, the pupil must be located. Relying on reflected light to find an object the size or shape of the pupil, however, does not ensure that the pupil is actually found. For example, a tear layer or flap bed can reflect light in a manner that causes these objects to expand and appear to be the size the pupil. Additionally, a flap bed can scatter light to create a circular pattern and thus appear to be both the size and shape of the pupil. Therefore, there is a need for a method and system to identify a pupil that accurately locates the pupil while reducing or eliminating false identifications.

### SUMMARY OF THE INVENTION

Embodiments of the present invention provide a method and system for automatically locating a pupil for tracking by an ophthalmic tracking system. Broadly speaking, an optical beam is scanned across the eye. In any given cycle, the reflected light from the eye is detected by a light sensor and data from the sensor processed to locate the position of a geometric feature of the reflected light, such as the centroid. If the distance between the position of the optical beam and the geometric feature is greater than a threshold value, the system determines that the pupil has been located.

One embodiment of the present invention includes a method for acquiring a pupil for use in tracking an eye by an eye tracking system comprising, directing an optical beam to an eye, receiving reflected light of the optical beam from the eye and determining a difference value representing a difference between a position of the optical beam and a position of a geometric feature of the reflected light. If the difference value is greater than a threshold, the method can include determining that the pupil has been located. If the difference value is not greater than the threshold, the method can include continuing to scan the eye with the optical beam. According to various embodiments of the present invention, the optical beam is an eye-safe beam, such as a 905 nanometer laser, and the geometric feature located is the centroid of the reflected light from the pupil.

Another embodiment of the present invention includes a refractive laser surgery system comprising a light source configured to generate an optical beam, a light sensor sensitive to light from the optical beam and one or more optical components configured to direct the optical beam to an eye and direct reflected light to the light sensor. The system can also comprise a controller configured to determine a position of a geometric feature of the reflected light based on data from the light sensor, determine a difference value representing a difference between the position of the geometric feature of the reflected light and a position of the optical beam and, if the difference value is greater than a threshold, determine that a pupil of the eye is located.

Yet another embodiment of the present invention includes a computer program product comprising a set of computer instructions stored on a computer readable medium. The set of computer instructions can comprise instructions executable by a processor to determine the position of a geometric feature of reflected light based on data from an optical sensor, determine a difference value representing a difference between the position of the geometric feature and a position of an optical beam, compare the difference value to a threshold and if the difference value is greater than the threshold, determine that a pupil of the eye is located.

Embodiments of the present invention provide a system and method for acquiring a pupil that is robust across all pupil sizes and changes that occur during the acquisition procedure (e.g., eye movement, dilation or other changes). The acquisition scheme does not require the pupil to be stable (e.g., dilated and paralyzed) to effectively locate the pupil.

### BRIEF DESCRIPTION OF THE FIGURES

A more complete understanding of the present invention and the advantages thereof may be acquired by referring to the following description, taken in conjunction with the accompanying drawings in which like reference numbers indicate like features and wherein:

FIG. 1 is a diagrammatic representation of one embodiment of a system for refractive laser surgery according to the present invention;

FIGS. 2A-2E are diagrammatic representations of scanning an optical beam across an eye and example reflection patterns detected by a light sensor according to the present invention;

FIG. 3 is a graph illustrating the relative distance between the position of the centroid of reflected light and the position of an optical beam according to the present invention; and

FIG. 4 is a flow chart illustrating one embodiment of a method of the present invention for acquiring the pupil.

### DETAILED DESCRIPTION

Preferred embodiments of the invention are illustrated in the FIGURES, like numerals being used to refer to like and corresponding parts of the various drawings.

A retroreflector reflects light back in the direction from which the light came. The human eye, because of its optical system and generally spherical shape, acts as a retroreflector to reflect light that enters the pupil back out of the pupil. This causes the frustrating “red eye” phenomenon in photographs. Red eye results when light from a flash is reflected off the blood rich retina back to the camera from which the flash emanated, causing the subject's pupils to appear red in the resulting photograph. A similar phenomenon, referred to as “pupil glow” results from the retroreflecting characteristics of the eye. When a relatively focused light enters the eye, the reflected light is spread over the entire area of the pupil and reflected back in the direction from which it came. Thus, the entire pupil appears to glow even if the source light is much smaller than the pupil.

Generally speaking, embodiments of the present invention utilize pupil glow to “find” the pupil (determine the position of the pupil) for an eye tracking system. An illumination light, such as a 905 nanometer laser, is scanned across the eye and the reflected light captured by a light sensor, such as a camera. When the illimination light reaches the edge of the pupil and enters the pupil, the entire pupil will appear to be illuminated, and the reflected light will have a relatively large size and generally circular shape. Additionally, the centroid of the reflected light will approximately correspond to the center of the pupil and therefore be offset from the position of the illumination beam on the eye. This offset will be approximately the radius of the pupil. Embodiments of the present invention can compare the location of the centroid of the reflected light (or location of another geometric feature of the reflected light) to the location of the illumination beam and, if the distance is greater than a predefined threshold, determine that the pupil has been located. Reflected light from tear layers, flap beds and other aberrations in the eye are not identified as the pupil because the centroid of the reflected light from these features will be at or near the location where the illumination light is aimed.

Embodiments of the present invention can be implemented with various eye tracking systems such as those disclosed in U.S. Pat. Nos. 6,302,879, 6,568,808, 6,626,896, 6,569,154, 7,044,944, 6,626,894, and 6,626,898, each of which is fully incorporated by reference herein, to locate the pupil of the eye. When the pupil is located, pupil based tracking of the eye movement can be utilized during a surgical procedure.

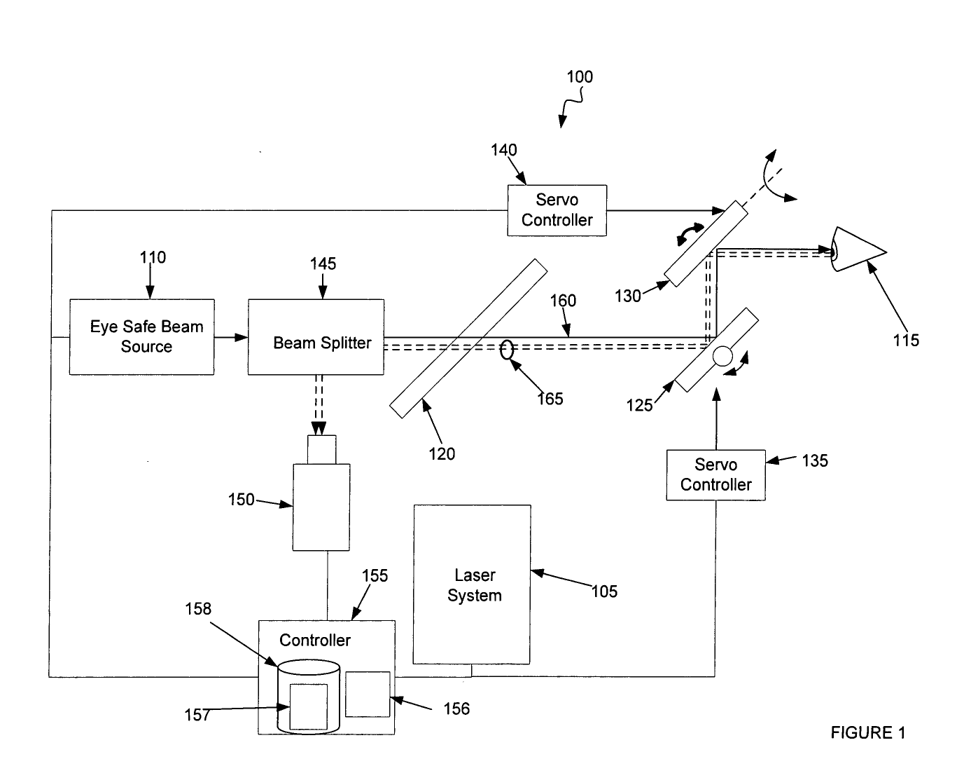

FIG. 1 is a diagrammatic representation of one embodiment of a refractive laser surgical system 100 that can utilize embodiments of the present invention. System 100 can include a laser system 105 which can include a laser source and optics (including projection optics and x-y translation optics) to project a laser beam to perform a surgical procedure. An example laser source is a 193 nanometer wavelength excimer laser used in ophthalmic PRK, LASEK, LASIK or other procedures. System 100 can also include a light source 110 that is preferably an eye-safe light source as defined by the American National Standards Institute (ANSI). According to one embodiment, light source 110 can be 100 microwatt, continuous wave laser. In another embodiment, light source 110 can be a high pulse repetition rate GaAs 905 nanometer laser operating at 4 kHz, which can produce, for example, a pulse of 10 nanojules in a 50 nanosecond pulse.

Light from laser system 105 and from light source 110 can be directed to eye 115 via a variety of beam splitters and mirrors. Beam splitter 120, according to one embodiment, can be a diachronic beam splitter that reflects light from laser system 100 to independently rotating mirrors 125 and 130 and allows light from light source 110 to pass to rotating mirrors 125 and 130. Servo controller 135 and servo controller 140 can manage servos to rotate mirrors 125 and 130, respectively, to direct beams of light generated by laser system 105 and light source 110 across eye 115. Light generated by light source 110 and reflected by eye 115 is directed by beam splitter 145 to light sensor 150. Beam splitter 145 can have any suitable configuration to allow light from light source 110 to pass and redirect light reflected by eye 115 to light sensor 150. For example, beam splitter 145 can be a mirror with a small hole in the center that allows the beam from light source 110 to pass, but has little affect on the image detected by light sensor 150. According to another embodiment of the present invention, light from light source 110 can be polarized and beam splitter 145 can be configured to transmit the polarized light. The light reflected by eye 115, however, is typically depolarized and beam splitter 145 can reflect the non-polarized light to light sensor 150.

System 100 is provided by way of context, but not limitation. System 100 can further include a variety of beam splitters and mirrors including, for example, beam splitters to direct light to/from other sensors and light sources used in eye tracking, OCT imaging systems or any other systems used to provide light to and detect light from eye 115. Embodiments of the present invention can be employed in any system configured to direct a light beam to an eye and direct the reflected light of the light beam from the eye to a light sensor sensitive to the reflected light. Thus, system 100 can include a variety of components to direct light from light source 110 to eye 115, scan light across eye 115 and direct reflected light to light sensor 150.

Controller 155 can include any suitable controller that can receive data from light sensor 150 and generate control signals to laser system 105, eye safe beam source 100, server controller 135 and servo controller 145. Controller 155 can include a processor 156, such as an ASIC, CPU or other processor and computer instructions 157 executable by the processor (e.g., software or other instructions stored on a computer readable medium). The instructions can be stored on a computer readable memory 158 (e.g., hard drive, Flash memory, optical memory, RAM, ROM or other computer readable medium known in the art). The processor 156 may be a single processing device or a plurality of processing devices. Such a processing device may be a microprocessor, micro-controller, digital signal processor, microcomputer, central processing unit, field programmable gate array, programmable logic device, state machine, logic circuitry, analog circuitry, digital circuitry, and/or any device that manipulates signals (analog and/or digital) based on operational instructions. The computer readable memory 158 may be a single memory device or a plurality of memory devices. Such a memory device may be a read-only memory, random access memory, volatile memory, non-volatile memory, static memory, dynamic memory, flash memory, cache memory, and/or any device that stores digital information. Note that when the processor 156 implements one or more of its functions via a state machine, analog circuitry, digital circuitry, and/or logic circuitry, the memory storing the corresponding operational instructions may be embedded within, or external to, the circuitry comprising the state machine, analog circuitry, digital circuitry, and/or logic circuitry. The computer readable memory 158 stores, and the processor 156 executes, operational instructions corresponding to at least some of the steps and/or functions illustrated in the FIGs. While controller 155 is shown as a single block in FIG. 1 for the sake of simplicity, the control functionality of system 100 can be distributed among multiple processors.

In operation, a beam of light 160 from light source 110 is directed to eye 115 according to any suitable mechanism including beam splitters (e.g., beam splitter 145), mirrors (e.g., mirrors 125 and 130) or other mechanisms for directing light to a target. Light beam 160 is scanned across the eye (e.g., by controlled movement of mirrors 125 and 130, movement of light source 110 or movement of eye 115 relative to light source 110) and the reflected light 165 from light beam 160 is directed to light sensor 150. The pattern of reflected light 165 detected by light sensor 150 is analyzed to determine the offset between the location of beam 160 and a feature of the reflected light. For example, the position of light beam 160 can be compared to the position of the centroid of the reflected light 165 to determine if the pupil has been located, as discussed below.

According to one embodiment of the present invention, light sensor 150 can be a CMOS camera sensitive to the infrared light of light beam 160 reflected by eye 115. When a pixel is illuminated, the camera outputs a voltage level proportional to the illumination of the pixel. According to one embodiment, controller 155 applies a threshold to the signal and if the voltage is sufficiently high, considers the pixel to be “on”. A minimum threshold can be applied so that pixels are not considered illuminated based on light that is too low in intensity. This can prevent falsely considering environmental light, light bleed, noise or other factors to be part of the reflected light 165. Controller 155 can create a binary representation of the image with pixels in the “on” state assigned a 1 and pixels in the off state (i.e., having a threshold below the minimum) assigned a 0. The position of the centroid of the reflected light 165 or other geometric feature of the reflected light 165 can be determined by analyzing the data from light sensor 150.

When light beam 160 enters the pupil near the edge of the pupil, light beam 160 will be retroreflected and scattered across the entire pupil. Thus, reflected light 165 will be distributed and have a centroid corresponding to the center of the pupil. Because the centroid of the reflected light 165 pattern approximately corresponds to the position of the center of the pupil rather than the position at which light beam 160 is aimed (i.e., the edge of the pupil), the centroid of reflected light 165 will be offset from the position of light beam 160. The offset between the position of the centroid of reflected light 165 and the position of light beam 160 on eye 115 can be used as an indication that the pupil has been located as other features of the eye 115 will not typically show a similar offset.

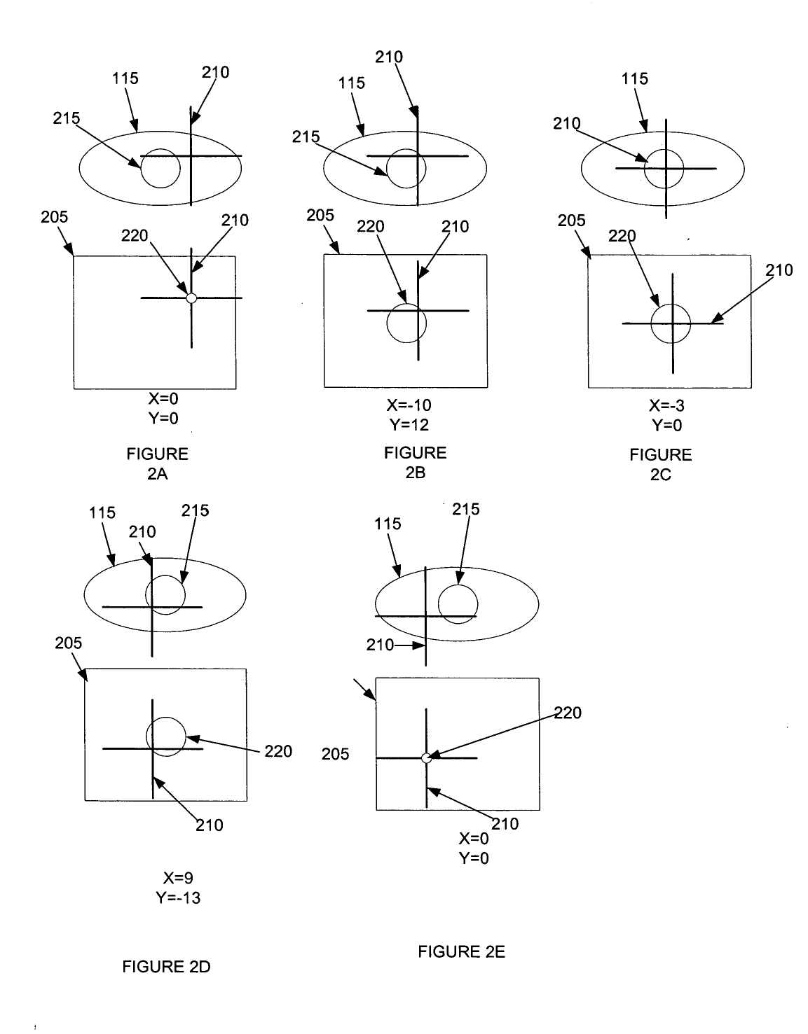

FIGS. 2A-2E are diagrammatic representations of eye 115 and a corresponding reflection pattern 205 generated by light sensor 150. According to one embodiment, eye 115 can be non-dilated and non-paralyzed during the pupil acquisition procedure. Crosshairs 210 represent the position of the illumination beam (e.g., beam 160) as it scans across eye 115 including pupil 215. Pixels 220 represent the pixels illuminated by light reflected by eye 115.

In FIG. 2A, illumination beam 160 impinges eye 115 away from pupil 215. As shown in reflection pattern 205, only a small number of pixels 220 are illuminated, roughly corresponding to the size of illumination beam 160. The centroid of the reflected light as detected by light sensor 150 approximately corresponds to the location at which illumination beam 160 is aimed. The position of the centroid of the reflected light and the position of light beam 160 can be mapped to an arbitrary value space (e.g., distance, Cartesian coordinates, pixel location, unit-less value) to determine the difference in position. In the example of FIG. 2A, the position of the centroid of the reflected light is mapped to coordinates having an origin at the position of light beam 160. Consequently, the centroid of the reflected light is at approximately 0,0. Thus, the measure of the difference between the location of illumination beam 160 and the centroid is approximately 0.

In FIG. 2B, on the other hand, illumination beam 160 impinges eye 115 at the edge of pupil 215 causing light to be reflected across the entire pupil, as represented by pixels 220. The centroid of the reflected light 165 as detected by light sensor 150 is offset from the location of illumination beam 160 (represented by the x=−10, y=12). Thus, the centroid is approximately 15.6 units from the position of the laser. This difference measure can optionally be converted into preferred units (e.g., millimeters or other units).

As the position of illumination beam 160 approaches the center of pupil 215, as shown in FIG. 2C, the offset between the centroid of the reflected light 165 and the position of light beam 160 becomes less. Relative to light beam 160, in this example, the centroid of the reflected light is at −3,0. That is, the centroid of the reflected light is only 3 units from the position of light beam 160. However, as shown in FIG. 2D, as the position of light beam 160 approaches the other edge of pupil 215, the difference between the position of the centroid of the reflected light 165 and the position of light beam 160 increases (e.g., in this example, the centroid of the reflected light is at −9,13 relative to the position of light beam 160). In FIG. 2E, illumination beam 160 has again moved off of pupil 215. In this example, the centroid of the reflected light detected by light sensor 150 is at approximately the position of illumination beam 160 (e.g., at 0,0).

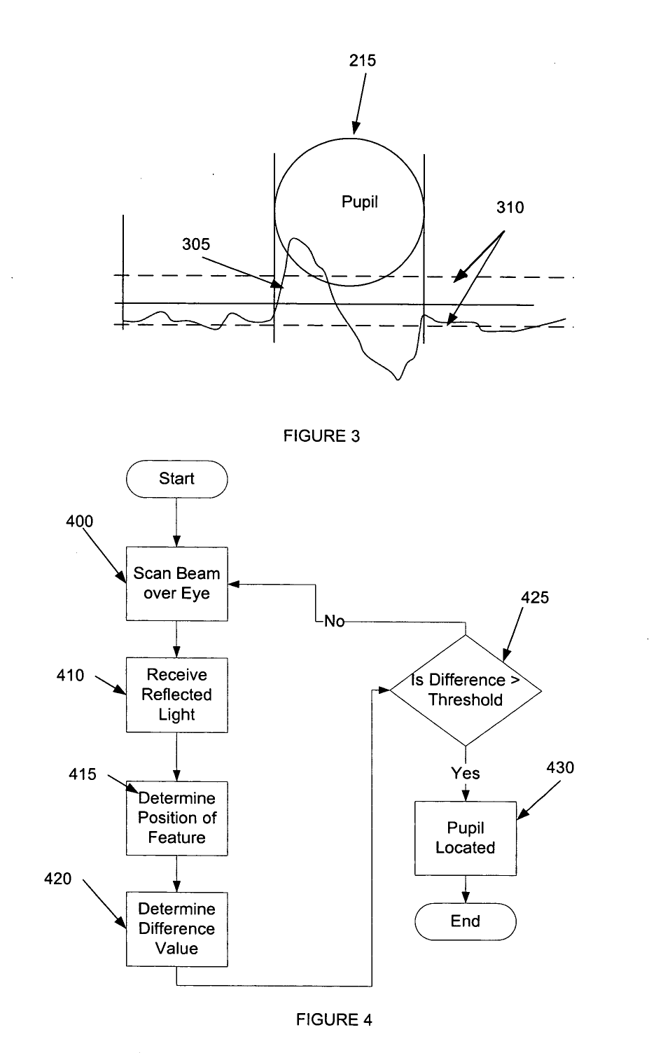

As can be understood from the foregoing examples, when beam 160 is near the edge of the pupil, the centroid of the reflected light 165 is offset from the position of beam 160 by a maximum amount due to pupil glow. When illumination beam 160 is aimed at the center of the pupil or at a feature of the eye that is not the pupil but that still reflects light, the centroid of the reflected light is close to the position of beam 160. FIG. 3 is a graph illustrating the difference measure between the position of illumination beam 160 and the centroid of reflected light as illumination beam 160 is scanned over eye 115, including pupil 215. Line 305 represents the difference between the center of the illumination beam 160 and the centroid of reflected light 165. As can be seen, when illumination beam 160 is near the edges of the pupil (going from left to right), the centroid has the greatest positive or negative offset from the position of illumination beam 160. At the center of pupil 215 and outside of pupil 215, the difference measure is approximately 0.

According to one embodiment, a threshold (represented by lines 310) can be applied to filter out differences below a certain amount. This can, for example, filter out noise from light sensor 150. Although shown as symmetrical, asymmetrical thresholds can be applied and other filtering techniques implemented. Any difference between the position of the centroid of the reflected light 165 registered by the light sensor 150 and the position of the illumination beam 160 that is outside of the threshold can indicate that the pupil has been located. Put another way, the system can determine that the pupil has been located if the distance between the centroid of the reflected light 165 and the position of the light beam 160 is greater than a predefined distance threshold.

FIG. 4 is a flow chart illustrating one embodiment of a method for determining the location of a pupil in accordance with the present invention. Various steps of FIG. 4 can be facilitated through the execution of computer instructions by a processor, such as processor 156 of FIG. 1. At step 400, an eye-safe illumination beam is scanned across the eye. The beam can have a diameter that is smaller than that of the typical pupil. For example, the illumination beam can have a diameter of approximately ⅓ mm, compared to a typical pupil size of 2 mm. Scanning can occur in any pattern and, according to one embodiment of the present invention, stop as soon as the pupil is located. The scanning pattern can be configured to be run in a short period of time (e.g., less than 0.5 seconds).

At step 410 the reflected light from the eye is detected and, at step 415, the position of a geometric feature of the reflected light is determined. For example, the location of the centroid of the reflected can be determined. At step 420 a value representing the difference between the location of the illumination beam and the geometric feature of the reflected light can be determined. If the value is greater than a predetermined threshold, as determined at step 425, the location of the pupil is considered to be found (step 430). Otherwise the method can return to step 400 and continue scanning the eye. In empirical tests with a 905 nanometer laser having a diameter of approximately ⅓ mm and using a threshold of 1 mm (i.e., so that the difference measure must correspond to a difference in position of greater than 1 mm), the pupil was correctly identified in over 99% of tests and no false positives were encountered. However, larger or smaller thresholds can be applied. The information regarding the location of the pupil or some feature of the pupil (e.g., the location of the center of the pupil) can then be used by other processes, such as eye tracking processes. The steps of FIG. 4 can be repeated as needed or desired.

While the present invention has been described with reference to particular embodiments, it should be understood that the embodiments are illustrative and that the scope of the invention is not limited to these embodiments. Many variations, modifications, additions and improvements to the embodiments described above are possible. It is contemplated that these variations, modifications, additions and improvements fall within the scope of the invention as detailed in the following claims.

What is claimed is:

1. A method for acquiring a pupil comprising:
directing an optical beam to an eye;
receiving reflected light of the optical beam from the eye; and
determining a difference value representing a difference between a position of the optical beam and a position of a geometric feature of the reflected light;
if the difference value is greater than a threshold, determining that the pupil has been located; and
if the difference value is not greater than the threshold, continuing to scan the eye with the optical beam.

2. The method of claim 1, wherein the geometric feature is the centroid of the reflected light.

3. The method of claim 2, further comprising determining the location of the centroid of the reflected light from data generated at a light sensor.

4. The method of claim 1, further comprising ending scanning of the eye with the optical beam when the pupil is located.

5. The method of claim 1, wherein the optical beam is an eye-safe optical beam.

6. The method of claim 5, wherein the optical beam is 905 nanometer laser beam.

7. The method of claim 1, further comprising leaving the eye untreated to achieve dilation and paralysis prior to directing the optical beam to the eye.

8. The method of claim 1, wherein the threshold corresponds to a distance of at least one millimeter.

9. A refractive laser surgery system comprising:
a light source configured to generate an optical beam;
a light sensor sensitive to light from the optical beam;
one or more optical components configured to:
direct the optical beam to an eye;
direct reflected light of the optical beam from the eye to the light sensor; and
a controller coupled to the light sensor configured to:
determine a position of a geometric feature of the reflected light based on data from the light sensor;
determine a difference value representing a difference between the position of the geometric feature of the reflected light and a position of the optical beam; and
if the difference value is greater than a threshold, determine that a pupil of the eye is located.

10. The refractive laser surgery system of claim 9, wherein the geometric feature is the centroid of the reflected light.

11. The refractive laser surgery system of claim 9, wherein the controller is configured to determine the centroid of the reflected light by analyzing which pixels of the light sensor are in an on state.

12. The refractive laser surgery system of claim 9, wherein the optical beam is an eye-safe optical beam.

13. The refractive laser surgery system of claim 12, wherein the eye-safe optical beam is a 905 nanometer laser.

14. The refractive laser surgery system of claim 9, wherein the threshold corresponds to a distance of greater than one millimeter.

15. A computer program product comprising a set of computer instructions stored on a computer readable medium, the set of computer instructions comprising instructions executable by a processor to:
determine the position of a geometric feature of reflected light based on data from an optical sensor;
determine a difference value representing a difference between the position of the geometric feature and a position of an optical beam;
compare the difference value to a threshold; and
if the difference value is greater than the threshold, determine that a pupil of the eye is located.

16. The computer program product of claim 15, wherein the geometric feature is a centroid of reflected light.

17. The computer program product of claim 15, wherein the threshold corresponds to a difference in position of at least 1 millimeter.
